(19) United States
(12) Patent Application Publication
(10) Pub. No.: US 2010/0176056 A1
(43) Pub. Date: Jul. 15, 2010

(54) METHOD FOR PREVENTING BIOFOULING ON SURFACES USING ULTRAVIOLET PRE-TREATMENT

(76) Inventor: Ytzhak Rozenberg, Ramat Gan (IL)

Correspondence Address: Pearl Cohen Zedek Latzer, LLP, 1500 Broadway, 12th Floor, New York, NY, 10036, US

(21) Appl. No.: 12/282,512

(22) PCT Filed: Jun. 15, 2008
(86) PCT No.: PCT/IL08/00811

§ 371 Date: Sep. 11, 2008

Related U.S. Application Data
(60) Provisional application No. 60/929,117, filed on Jun. 13, 2007.

(51) Int. Cl. A61L 2/10; C02F 1/32; C02F 1/44

(52) U.S. Cl. 210/636; 210/748.1; 210/748.11

(57) ABSTRACT

A method for reducing bio-fouling formation on a liquid-contacting surface by pre-designed pretreatment of the liquid with UV light is provided. The method may include flowing liquid through a conduit having ultraviolet-transparent walls; exposing the liquid within the conduit to ultraviolet (UV) light rays from a polychromatic light source that emits in the spectral band of 200 nm to 300 nm and produces effective UV-Dose of above 40 mJ/cm² such that a portion of the UV light rays is reflected back into the liquid by total internal reflection. After exposure to the UV light, the liquid is contacted the liquid-contacting surface, for example a reverse osmosis membrane without causing biofilm formation.

### CROSS-REFERENCE TO RELATED APPLICATIONS

This application is a National Phase Application of PCT International Application No. PCT/IL2008/000811, International Filing Date Jun. 15, 2008, claiming priority of U.S. Provisional Patent Application 60/929,117, filed Jun. 13, 2007 the entire disclosure of which is incorporated herein by reference.

### BACKGROUND OF INVENTION

The formation of biofilms onto water filtering membranes is a major problem in water-related processes and particularly in water desalination processes. The membranes are susceptible to clogging by waterborne micro-organisms, which leads to biofouling of the membrane surface and results in reduced membrane performance. The problem of biofouling of membranes, pipes, cooling towers and other water-contacting surfaces has not been successfully dealt with existing solutions.

Biofouling or biological fouling is the undesirable accumulation of microorganisms, plants, algae, and micro-animals on submerged structures. Biofilms are formed when bacteria adhere to a hard surface in an aqueous environment. Over a period of time, microbes, entering the pipe stick to an already existing bacterial layer thereby forming a microbial matrix. This matrix, once established, supplies nutrients required for growing additional microbial mass.

Possible solutions are use of disinfecting material such as chlorine or use of carbon black filters. Chlorine is the most widely used disinfectant for inactivating (“killing”) microorganisms in water and slowing or preventing the formation of biofilms. However, disinfection treatments with chlorine can produce a wide variety of by-products, many of which have been shown to cause cancer and other toxic effects. Further, some of the surfaces which are in contact with disinfecting gents, such as membranes, may be damaged by the inorganic disinfecting treatment.

While carbon black filters have been somewhat effective in removal of organics and inorganic materials, these carbon filters are incapable of removing microorganisms such as bacteria, viruses, yeasts or molds and may spread carbon particles in the system al filters.

Accordingly, there is a need for a process for controlling biofilm in a water line, which will minimize the risk that a user of the water line will be exposed to a residual disinfectant. UV-based liquid disinfection systems are known. Ideally, UV-based disinfection systems should be constructed such that each microorganism crossing the system is irradiated with a uniform UV dose. Existing disinfection systems do not have the capability of effectively achieving the delivery of UV dose to substantially all microorganisms crossing the system.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings in which:

FIG. 1 is a conceptual illustration of an anti-fouling system according to embodiments of the invention;

FIG. 2 is a conceptual illustration of a UV liquid disinfection system capable of slowing the process of bio-fouling of liquid-contacting surfaces according to embodiments of the invention;

FIG. 3 is a conceptual illustration of a UV liquid disinfection system capable of slowing the process of bio-fouling of liquid-contacting surfaces according to embodiments of the invention;

FIG. 4 represents a fluorescent analysis of bio-film formation or prevention on RO membranes helpful in demonstrating embodiments of the invention;

FIG. 5 is a photograph showing bio-film formation or bio-film prevention on RO membranes helpful in demonstrating embodiments of the invention; and

FIG. 6 is a graph representing microbiological analysis of reverse osmosis helpful in demonstrating embodiments of the invention.

It will be appreciated that for simplicity and clarity of illustration, elements shown in the drawings have not necessarily been drawn accurately or to scale. For example, the dimensions of some of the elements may be exaggerated relative to other elements for clarity or several physical components included in one functional block or element. Further, where considered appropriate, reference numerals may be repeated among the drawings to indicate corresponding or analogous elements. Moreover, some of the blocks depicted in the drawings may be combined into a single function.

### DETAILED DESCRIPTION OF EMBODIMENTS OF THE INVENTION

In the following detailed description, numerous specific details are set forth in order to provide a thorough understanding of the invention. However, it will be understood by those of ordinary skill in the art that the present invention may be practiced without these specific details. In other instances, well-known methods, procedures and components may not have been described in detail so as not to obscure the present invention.

The term “anti-biofouling”, throughout the Specification and claims refers to the process of preventing or diminishing accumulation of microorganisms on surfaces of any structure that is in contact with liquid. In other words, the term “anti-biofouling” refers hereinafter to preventing or reducing bio-fouling.

“Micro-organism” (also spelled as “microorganism”) or “microbe” is an organism that is microscopic and can be bacteria, fungi, archaea or protists. Micro-organisms are generally single-celled, or unicellular organisms; however, there are exceptions as some unicellular protists are visible to the average human, and some multicellular species are microscopic. Microbiological organisms commonly found within industrial water systems which thus far have been detectable by and respond to the detection methods of the present process include, but are not limited to, Pseudomonas, Bacillus, Klebsiella, Enterobacter, Escherichia, Sphaerotilus and Haliscomenobacter.

Some demonstrative embodiments of the invention may include a method of slowing the process of biofouling formation on liquid-contacting surfaces by pre-treating the liquid. The exposure of the liquid to UV light at a predetermined UV dose may eliminate or prevents biofilm formation on the surface. Non-limiting examples of surfaces prone to biofouling formation such as reverse osmosis membranes used for example for in water desalination processes or any other membranes, external surfaces of pipes, external surfaces of containers and others.

The method may include passing the liquid through an ultraviolet (UV) liquid disinfection device as described in detail below. It will be appreciated that the term “liquid” as used herein may refer to any liquid, e.g., including water or water-based liquid intended to be disinfected.

According to embodiments of the invention, the UV disinfection pretreatment may include flowing liquid through a conduit having ultraviolet-transparent walls and exposing the liquid within the conduit to ultraviolet (UV) light rays from a polychromatic light source that emits in the spectral band of 200 nm to 300 nm and effective UV-Dose above 40 mJ/cm2 such that a portion of the UV light rays is reflected back into the liquid by total internal reflection. After exposure to the UV light, the liquid may be contacted with the liquid-contacting surface.

According to exemplary embodiments of the invention, the conduit may be made, at least partially, of a UV transparent material, such as quartz and the UV source may be located externally to the UV-transparent conduit adjacent a UV-transparent window capable of transmitting light emitted from the UV source. The liquid within the conduit may act as a waveguide enabling at least part of the UV light emitted from the UV source to be totally-internally reflected at the interface of the UV-transparent conduit and the air surrounding it. The UV source may be a medium pressure UV lamp. According to some embodiments the UV source may emit in the spectral band of 200 nm to 300 nm. The UV source may emit light such that at least 15% of the total power from the light source is light having a wavelength between 200 nm to 250 nm

Alternatively, the UV source may be surrounded by a UV-transparent sleeve positioned within the conduit substantially perpendicular to the axis of symmetry of the conduit. In such a configuration, the liquid within the conduit may act as a waveguide and at least part of the light emitted from the UV light source may be totally-internally reflected at the interface of the conduit and the air surrounding it.

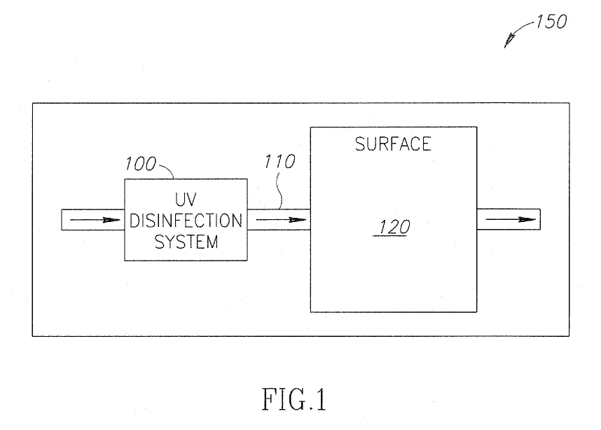

Reference is now made to FIG. 1 which conceptually illustrates an anti-fouling UV liquid disinfection system for pretreatment of liquids according to embodiments of the invention. An antifouling system 500 may include a UV disinfection system 100, conveying system 200 and a structure having a surface 300 prone to biofouling. In some embodiments of the invention, there is provided a method for preventing or diminishing biofouling on a structure surface, such as a membrane which come into contact with liquid by delivering the liquid via UV disinfection system 100 prior to the contacting the liquid with the surface 300.

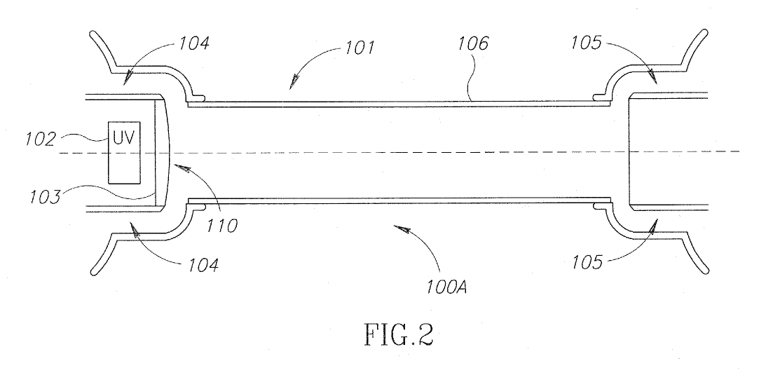

For example, as illustrated in FIG. 2, a UV disinfection system 100A may include a conduit with transparent walls and at least one UV source located outside the conduit proximate to a transmmitive window, such as for example, R200DL/SL, manufactured by Atlantium Technologies Ltd. of Har-Tuv, Israel. According to some demonstrative embodiments of the invention, system 100A may include a conduit 101 to carry a flowing liquid to be disinfected and an external UV source 102 to illuminate the liquid within conduit 101. Although the invention is not limited in this respect, illumination source 102 may generate UV light of a suitable UV spectrum. Conduit 101 may have an inlet 104 to receive the liquid, and an outlet 105 to discharge the liquid. Conduit 101 may further include walls 106 may be made of transparent material, such as quartz, and a UV-transparent window 110 located at the end of conduit 101 proximate to UV source 102. The light produced by UV source 102 may be directed toward the liquid within conduit 101 via window 110. Window 110 may be made of quartz. Any other suitable UV-transparent material may be used.

In the exemplary illustration of FIG. 2 the window-lamp assembly is illustrated as being in proximity to the water inlet. It should, however, be understood to a person skilled in the art that embodiments of the invention are not limited in this respect and alternatively or additionally, the window-lamp assembly may be positioned in proximity to the outlet of the conduit. According to another embodiment of the present invention, system 100A may include two windows, each at one end of conduit 101.

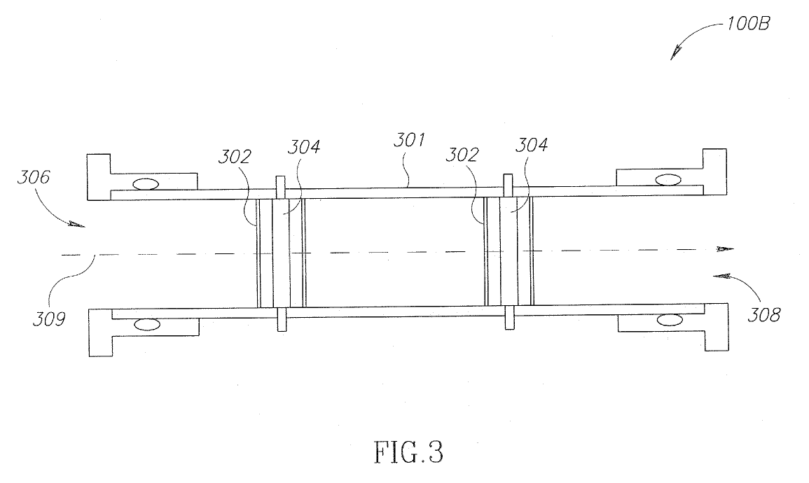

Alternatively, as illustrated in FIG. 3, a UV disinfection system 100B may include a conduit with transparent walls and at least one UV source located inside the conduit in a transimmisive sleeve perpendicular to the direction of flow of the liquid, such as for example, RZ104-xy, manufactured by Atlantium Technologies Ltd. OF Har-Tuv, Israel. System 100B may include a conduit 301 made of substantially UV-transparent glass, such as quartz to carry liquid to be disinfected. System 100B may further include one or more substantially UV-transparent sleeves 302 positioned within conduit 301 substantially perpendicular to its longitudinal axis of symmetry. A non-exhaustive list of suitable UV-transparent materials for the sleeve may be quartz or Teflon. Both ends of sleeve 302 extend from the walls of the conduit to enable the insertion of a UV source 304 within sleeve 302. UV sources 304 may illuminate the liquid to be disinfected when flowing in the conduit. Conduit 301 may have an inlet 306 to receive the liquid to be disinfected and an outlet 308 to discharge the liquid.

In the configuration described above, in which the UV source 304 is positioned substantially perpendicular to the longitudinal axis of symmetry 309 of conduit 301 and to the direction of flow of the liquid, the liquid may act as a waveguide and at least part of the radiation may be totally-internally reflected at the interface of the glass conduit and air surrounding it.

In both configurations, the liquid within the conduit may act as a waveguide and at least part of the light emitted from the UV light source may be totally-internally reflected at the interface of the conduit and the air surrounding it. It should be understood to a person of ordinary skill in the art that the embodiments of disinfection system 100 described above do not limit the invention in this respect and any other UV disinfection system capable of emitting light at a uniform UV dose above a desired level may be used. The term hydro-optic disinfection (HOD) system used herein refers to such system capable of reflecting at least a portion of the UV light by total internal reflection.

According to embodiments of the invention, system 100 may be capable of emitting UV is such a way as to produce a narrow dose distribution, defined as the average UV dose minus the minimum UV dose divided by the average UV dose. According to embodiments of the invention, the UV dose distribution may be lees than 0.5.

According to embodiments of the invention, UV source 102 and 304 may be a broad-band light source, such as medium-pressure UV-lamps. The UV source may emit at least 15% of the total input electrical power as light power in the range of 200 nm to 300 nm (the “germicidal” range) spectrally spread such that at least 15% of the light power are in the range of 200 nm to 250 nm.

According to embodiments of the invention, the average velocity of the liquid flowing through system 100 may be equal or above approximately 0.25 meter/second. System 100 and conduits 101, 301 may have relatively small cross-sectional dimensions. For example, the smallest cross-sectional plane dimension of the conduit, essentially transverse to the liquid flow direction may be less then 20 cm. Therefore, if the cross-section plane of the conduit is circular, than the diameter of the conduit may not exceed approximately 20 cm.

In some embodiments of the invention, there is provided a method of anti-biofouling by treating the liquid prior to contacting the surface such that the UV dose to each waterborne microorganism crossing the system is substantially similar.

Following are exemplary processes demonstrating embodiments of the present invention in a reverse osmosis membrane.

### Example 1

### Experimental Procedures

A first reverse osmosis (RO) membrane was inserted to receive liquid exiting a UV disinfection system commercially known as R200-DL and manufactured by Atlantium Technologies Ltd. The first RO membrane is also termed herein as Post-HOD membrane. A second reverse osmosis membrane was inserted near the inlet of the UV disinfection system. The second RO membrane is also termed herein as Per-HOD membrane. The water flow was 70 m³/hr and the average UV doze was around 100 mJ/cm². After 17 days the membranes were removed from their positions and inspected.

Three randomly selected samples were cut from each membrane, fixated by paraformaldehyde (4% for 15 minutes at room temperature) and rinsed twice by iso-normal PBS (pH 7.4). The samples were then labeled by Propidium Iodide (2 μg/ml in PBS for 15 minutes at room temperature) and rinsed by PBS three times. It is noted that Propidium Iodide indicates cells viability by binding to DNA in to produce orange-red fluorescence centered at 617 nanometers. The positively charged fluorophore also has a high affinity for double-stranded RNA. Propidium has an absorption maximum at 536 nanometers, and can be excited by the 488-nanometer or 514-nanometer spectral lines of an argon-ion (or krypton-argon) laser, or the 543-nanometer line from a green helium-neon laser.

Five different points were examined from each membrane samples by LSM confocal microscope (Zeiss LSM-410) using 488 and 533 excitation filters. The preparations were optically sliced (five slices of 10 μm each) to depth of 52-58 μm and photographed to fluorescent visual images. The PI fluorescence was analyzed using ImagePro+ software.

### Experimental Results

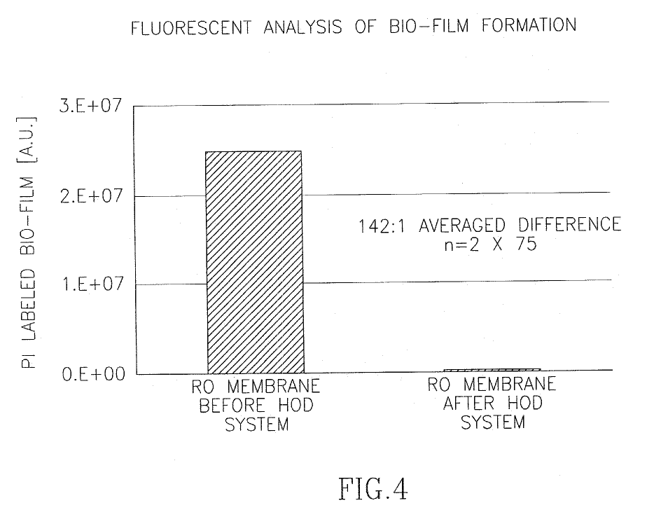

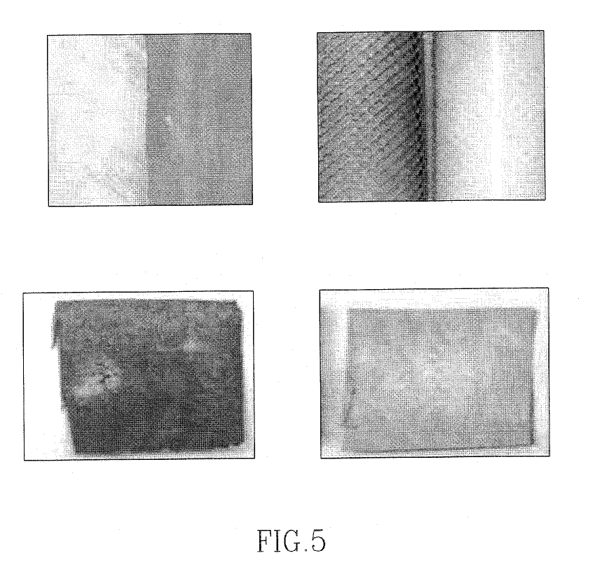

FIG. 4 is a graph demonstrating the fluorescent analysis of the PI labeled Pre-HOD membrane and PI labeled Post-HOD membrane. As can be seen, the PI labeled for the Pre-HOD membrane was 142 times higher than the Post-HOD membrane (A.U. in the graph stands for Arbitrary Units). FIG. 5 shows a photographic comparison between Pre-HOD and Post-Hod membranes. In each photograph, the left side shows a Pre-HOD membrane that received untreated liquid and therefore biofilm was formed on the membrane and the right side shows a Post-HOD membrane that received treated liquid after UV disinfection pretreatment. As can be seen the Post-Hod membranes have a lighter color that the Pre-Hod membrane since substantially no biofilm was formed on the Post-Hod membrane.

These results clearly show that the biofouling was dramatically reduced in membranes treated according to the method of the present invention and strengthen the use thereof for preventing or reducing biofouling in surface of structures that come in contact with aquatic environments and in particular in membranes.

It will be appreciated by persons skilled in the art that the present invention is not limited by what has been particularly shown and described herein above. Rather the scope of the present invention includes both combinations and subcombinations of the various features described hereinabove as well as variations and modifications which would occur to persons skilled in the art upon reading the specifications and which are not in the prior art.

### Example 2

The physical properties of two new reverse osmosis membranes, the first related to herein as Membrane A, and the second related to herein as Membrane B, were tested using tap water. The physical results of those tests are shown in Tables I and II, where Table I comprises the physical properties of Membrane A, while Table II comprises the physical properties of Membrane B.

Membrane A was placed in a brackish water desalination system, where the water was treated by UV light using the Hydro-Optic-Disinfection (“HOD”) system, which, as detailed above, enables total internal reflection (TIR) before reaching Membrane A.

Membrane B was also placed in the same brackish water desalination system, however, the water reaching Membrane B was not treated by UV light.

Water was allowed to circulate through the desalination system for 30 days, during which all of the water directed to Membrane A was first treated by the HOD system, while all of the water directed to Membrane B received no such treatment.

After 30 days Membranes A and B were removed from the desalination system, and their physical properties were tested using the same testing procedures as were used for the same membranes before their placement in the desalination system. The physical results of Membranes A and B, 30 days after they were placed in the desalination system, are shown in Tables III and IV, where Table III comprises the physical properties of Membrane A, while Table IV comprises the physical properties of Membrane B.

When comparing the results in Tables I and III it appears that the flux rate of Membrane A remains constant at 12.95 L/(m² h) before and after treating brackish water for 30 days. Thus, Membrane A shows no signs of biofouling. In contrast, when comparing the results in Tables II and IV it appears that the flux rate of Membrane B drops from 15 L/(m² h), before treating brackish water, to 10.23 L/(m² h), after treating brackish water for 30 days. The drop in the flux rate of Membrane B teaches of a relatively high degree of biofouling.

Since Membranes A and B were both placed in the same desalination system, both treating the same amount of water for the same length of time, the difference between the biofouling of the two membranes must be a result of the UV treatment of the water before reaching Membrane A using the HOD system of this invention.

It is noted that the initial flux rate of the two membranes, before treating the brackish water was expected to be identical; however, membrane A showed an initial flux rate of 12.95 L/(m² h), while Membrane B showed an initial flux rate of 15 L/(m² h). This teaches that the membranes are not initially identical; however this does not change the conclusions regarding the biofouling of Membrane B in contrast to the absence of biofouling of Membrane A.

TABLE 1
Membrane A before desalination

| Parameter | Unit | result |
|---|---|---|
| water type | | tap water |
| pressure | bar | 6 |
| membrane nominal area | L/(m²hr) | 0.44 |
| Permeate flow rate | lit/hr | 5.7 |
| Concentrate Flow rate | lit/hr | 99.96 |
| raw water conductivity | μs/cm | 1225 |
| concentrate conductivity | μs/cm | 1337 |
| permeate conductivity | μs/cm | 39.7 |
| water temp | ° c. | 24.8 |
| flux rate | L/(m²hr) | 12.95 |
| recovery | % | 5.394662 |

TABLE 2
Membrane B before desalination

| Parameter | Unit | result |
|---|---|---|
| water type | | tap water |
| pressure | bar | 6 |
| membrane nominal area | L/(m²hr) | 0.44 |
| Permeate flow rate | lit/hr | 6.6 |
| Concentrate Flow rate | lit/hr | 99.96 |
| raw water conductivity | μs/cm | 1225 |
| concentrate conductivity | μs/cm | 1337 |
| permeate conductivity | μs/cm | 39.7 |
| water temp | ° c. | 25.8 |
| flux rate | L/(m²hr) | 15 |
| recovery | % | 6.193694 |

TABLE 3
Membrane A after desalination (with UV)

| Parameter | Unit | result |
|---|---|---|
| water type | | tap water |
| pressure | bar | 6 |
| membrane nominal area | L/(m²hr) | 0.44 |
| Permeate flow rate | lit/hr | 5.7 |
| Concentrate Flow rate | lit/hr | 138 |
| raw water conductivity | μs/cm | 1220 |
| concentrate conductivity | μs/cm | 1240 |
| permeate conductivity | μs/cm | 50 |
| water temp | ° c. | 24.6 |
| flux rate | L/(m²hr) | 12.95 |
| recovery | % | 3.9665971 |

TABLE 4
Membrane B after desalination (without UV)

| Parameter | Unit | Result |
|---|---|---|
| water type | | tap water |
| pressure | bar | 6 |
| membrane nominal area | L/(m²hr) | 0.44 |
| Permeate flow rate | lit/hr | 4.5 |
| Concentrate Flow rate | lit/hr | 142.2 |
| raw water conductivity | μs/cm | 1220 |
| concentrate conductivity | μs/cm | 1240 |
| permeate conductivity | μs/cm | 50 |
| water temp | ° c. | 25.8 |
| flux rate | L/(m²hr) | 10.23 |
| recovery | % | 3.0674847 |

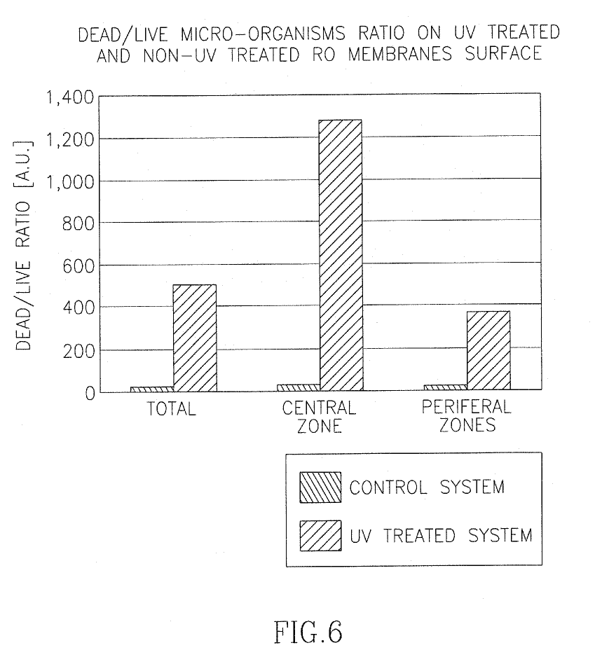

The results were further supported by microbiological studies. For Example FIG. 6 shows the ratio between dead and live microorganisms for both Pre-HOD and Post HOD membranes. The populations in the Pre-HOD and Post HOD membranes were different. The membrane that received liquid with UV from a HOD system had a larger ratio of dead to live microorganisms than the Pre-HOD membrane. Further a molecular analysis of microbial populations on both membranes was conducted. It appears that in the Pre-HOD membrane the Nitrospira species constitute that majority of the microbial population and further Brucellaceae bacteria that indicates the existence of biofouling was found. This type of microorganism is capable of serving as a good substrate for biofouling formation. In the Post-Hod membrane that received treated water the population was different. No Brucellaceae was found. Rather Gamma proteobacteria that was not found in the Pre-HOD membrane was detected here. In general, it appears that the population shifted towards Gamma proteobacteria. These results suggests that liquid pre-treatment by a UV disinfection system as described above can significantly reduce biofilm formation o RO membranes. This may be achieved by shifting the bacterial population towards microorganisms that are less capable of biofilm formation or less suited to thrive in the environmental conditions existing I the inner space of the reverse osmosis membranes

While certain features of the invention have been illustrated and described herein, many modifications, substitutions, changes, and equivalents may occur to those of ordinary skill in the art. It is, therefore, to be understood that the appended claims are intended to cover all such modifications and changes as fall within the true spirit of the invention. The results were further supported by microbiological studies.

What is claimed is:

1. A method for reducing the rate of bio-fouling formation on a liquid-contacting surface, the method comprising:
flowing liquid through a conduit, the conduit having ultraviolet-transparent walls;
exposing the liquid within the conduit to ultraviolet (UV) light rays from a polychromatic light source that emits in the spectral band of 200 nm to 300 nm and effective UV-Dose above 40 mJ/cm² such that a portion of the UV light rays is reflected back into the liquid by total internal reflection;
after exposure to the UV light, contacting the liquid with the liquid-contacting surface,
wherein the exposure to UV light slows or prevents biofilm formation on the surface.

2. The method of claim 1, wherein the liquid-contacting surface is a reverse osmosis membrane for desalination of the liquid and contacting the liquid with the membrane include passing the liquid trough the membrane.

3. The method of claim 1, wherein at least 15% of the total power from the light source is between 200 nm to 250 nm.

4. The method of claim 1, wherein exposing the liquid to UV light changes microbial populations within the liquid such that the number of microorganisms capable of biofilm formation is reduced.
